(19) United States
(12) Patent Application Publication
(10) Pub. No.: US 2023/0354820 A1
(43) Pub. Date: Nov. 9, 2023

(54) ANTI-PEST TOXINS AND CELLS

(71) Applicants: THE STATE OF ISRAEL, MINISTRY OF AGRICULTURE & RURAL DEVELOPMENT AGRICULTURAL, B, Rishon Lezion (IL); B. G. NEGEV TECHNOLOGIES AND APPLICATIONS LTD., AT BEN-GURION UNIVERSITY, Beer Sheva (IL); SAPIR ACADEMIC COLLEGE, Hof Ashkelon (IL); ACHVA ACADEMIC COLLEGE, Shikmim (IL)

(72) Inventors: Dana MENT, Nes-Ziona (IL); Zvi MENDEL, Tel Aviv (IL); Arieh ZARITSKY, Lehavim (IL); Ariel KUSHMARO, Beer Yaacov (IL); Zeev BARAK, Beer-Sheva (IL); Yaron SITRIT, Lehavim (IL); Eitan BEN-DOV, Beer Sheva (IL); Lilach Lily MONDACA, Kibbuts Or-haner (IL)

(21) Appl. No.: 18/217,660

(22) Filed: Jul. 3, 2023

Related U.S. Application Data
(63) Continuation-in-part of application No. PCT/IL2022/050015, filed on Jan. 4, 2022.
(60) Provisional application No. 63/133,437, filed on Jan. 4, 2021.

(51) Int. Cl. A01N 63/50; C07K 14/325; A01N 63/23; C12N 9/42; A01P 7/04

(52) U.S. Cl. CPC A01N 63/50; A01N 63/23; A01P 7/04; C07K 14/325; C12N 9/2442; C12Y 302/01014; C07K 2319/55

(57) ABSTRACT

The present invention is directed to a pesticide composition comprising at least two proteins selected from the group consisting of: CrylIa10, Cry2Abl, Cry9Eal, and the amino-acid sequence set forth in SEQ ID NO: 6. Further provided are the nucleic acid construct comprising a nucleic-acid sequence encoding CrylIa10, Cry2Abl, Cry9Eal, and the amino-acid sequence set forth in SEQ ID NO: 6, and a method of controlling a pest, comprising contacting the pest with an effective amount of the pesticide composition disclosed herein.

### CROSS REFERENCE TO RELATED APPLICATIONS

This application is continuation-in-part CIP of International Application No. PCT/IL2022/050015 filed Jan. 4, 2022, which claims the benefit of priority of U.S. Provisional Pat. Application No. 63/133,437, filed Jan. 4, 2021, the contents of which are all incorporated herein by reference in their entirety.

### SEQUENCE LISTING STATEMENT

The instant application contains a Sequence Listing which has been submitted electronically in ST26 format and is hereby incorporated by reference in its entirety. Said ST26 copy, created on Jun. 27, 2023, is named 1917-B-03-PCT-CIP SQL ST26.xml and is 31,056 bytes in size.

### FIELD

The disclosure relates in general to protein toxins, their encoding genes, various combinations thereof, cells comprising them, and their use in controlling pests.

### BACKGROUND

To control pests, farmers mostly rely on non-specific synthetic insecticides such as organophosphates, carbamates and neonicotinoids, however continuous exposure leads to the occurrence of highly resistant populations. Despite intensive applications of insecticides, significant economic losses caused by pests are still a major problem. The need for safe and effective management options for pest control are thus urgent and critical.

The entomopathogenic bacterium Bacillus thuringiensis (Bt) was first discovered over a century ago and has now become the leading biological insecticide used commercially to control insects. It is a gram-positive, aerobic, endospore-forming saprophyte species, naturally occurring in various soil and aquatic habitats. Various subspecies are recognized by their ability to produce large quantities of insect larvicidal “Cry” (for crystal) and “Cyt” (for cytolytic) -endotoxins assembled as parasporal crystalline bodies. These Insecticidal Crystal Proteins (ICPs), synthesized during sporulation, are tightly packed by hydrophobic bonds and disulfide bridges. The crystals are ingested by the pest larvae, solubilized in the insect midgut, and the proteolytically-activated ICPs insert into the apical microvilli membranes.

Development of Cry toxin-based biopesticides has relied on screening of natural isolates of Bt for toxins with activities against target pests. This approach identified many Cry toxins used to control agriculturally important pests. The high potencies and specificities ofICPs have spurred their use as natural control agents against insect pests in agriculture, forestry and human health.

The need for safe and effective management options for pests is thus urgent and critical.

### SUMMARY

According to a first aspect, there is provided a pesticide composition comprising at least two proteins selected from the group consisting of: Cry1Ia10, Cry2Ab1, Cry9Ea1, and the amino-acid sequence set forth in SEQ ID NO: 6 (M100 Vip3Aa79).

In some embodiments, the pesticide composition comprises CrylIa10, Cry2Ab1, Cry9Ea1, and the amino-acid sequence set forth in SEQ ID NO: 6.

In some embodiments, the pesticide composition further comprises at least one protein selected from the group consisting of: Chitinase, and Endochitinase.

In some embodiments, the pesticide composition further comprises Chitinase, and Endochitinase.

According to another aspect, there is provided a nucleic-acid construct comprising a nucleic-acid sequence encoding at least two proteins selected from: CrylIa10, Cry2Ab1, Cry9Ea1, and the amino-acid sequence set forth in SEQ ID NO: 6.

In some embodiments, the nucleic acid construct comprises a nucleic-acid sequence encoding CrylIa10, Cry2Ab1, Cry9Ea1, and a nucleic-acid sequence encoding the amino-acid sequence set forth in SEQ ID NO: 6.

In some embodiments, the nucleic-acid sequence encoding the amino-acid sequence set forth in SEQ ID NO: 6, comprises the nucleic-acid sequence set forth in SEQ ID NO: 12.

In some embodiments, the nucleic acid construct further comprises a nucleic-acid sequence encoding Chitinase, Endochitinase, and both.

In some embodiments, the pesticide composition comprises a bacterial cell or portion thereof.

In some embodiments, the portion thereof is selected from the group consisting of: an endospore, a crystal protein, an endospore-crystal protein complex, and any combination thereof.

In some embodiments, the bacterial cell belongs to Bacillus Thuringiensis specie.

In some embodiments, the bacterial cell is M100 strain of the Bacillus Thuringiensis specie.

According to another aspect, there is provided a method of controlling a pest, the method comprising steps of contacting the pest with an effective amount of the pesticide composition comprising at least two proteins selected from the group consisting of: CrylIa10, Cry2Ab1, Cry9Ea1, and the amino-acid sequence set forth in SEQ ID NO: 6 (M100 Vip3Aa79).

In some embodiments, the composition comprises CrylIa10, Cry2Ab1, Cry9Ea1, the amino-acid sequence set forth in SEQ ID NO: 6 (M100 Vip3Aa79), Chitinase, and Endochitinase.

In some embodiments, the pest is a lepidopteran pest.

In some embodiments, the pest is selected from the group consisting of: False codling moth, Carob moth, Darkling beetle, Spodoptera littoralis, Pine processionary moth, Pistachio processionary moth, Spodoptera Frugiperda, Spodoptera eridania, Heliothis virescens, Plutella xylostella, and Tuta absoluta.

In some embodiments, the effective amount is in the range of 500-160,000 ITU/mg.

In some embodiments, contacting is for less than 4 days.

Further embodiments, features, advantages and the full scope of applicability of the present invention will become apparent from the detailed description and drawings given hereinafter. However, it should be understood that the detailed description, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 illustrates the phylogenetic relationships (unrooted tree) of 40 Bacillus thuringiensis genomic sequences, including isolates M98 and M100.

FIG. 2 is a line graph demonstrating the effect of BTM and BTK standard products on the mortality (%) of Spodoptera frugiperda larvae during 4 days post inoculation.

FIG. 3 is a bar graph demonstrating the effect of BTM and BTK standard products on the damage index of tomato plants by Tuta absoluta after 7, 14, and 28 treatment days.

### DETAILED DESCRIPTION

The present invention is based on the surprising finding that certain newly-identified cells produce newly-identified proteins having anti-pest activity, thus considered anti-pest cells and anti-pest protein toxins, respectively.

The present invention provides, in one aspect, a protein, the protein comprising the amino acid sequence set forth in any one of SEQ ID NO: I (M98 Cry1Bk1), SEQ ID NO: 2 (M98 Cryi1a43), SEQ ID NO: 3 (M98 Cry2AbX), SEQ ID NO: 4 (M98 Vip3Aa80), SEQ ID NO: 5 (M98 Vpb1Ac2), SEQ ID NO: 6 (MIOO Vip3Aa79).

A person of the art would understand that such a protein includes, but is not limited to, a single protein which comprises a single amino-acid sequence of the indicated amino-acid sequences, a single protein which comprises different indicated amino-acid sequences, different proteins which comprise different indicated amino-acid sequences, and pluralities thereof.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, isolated protein, homologs, orthologs, paralogs, fragments and other equivalents, variants, fragments, and analogs of the foregoing. Polypeptides and proteins considered in the present invention are entire proteins or at least a sufficient portion of the entire protein to impart the relevant biological activity of the protein.

The amino acid sequence of the polypeptides disclosed herein can be identical to the wildtype sequences of appropriate components. Alternatively, any of the components can contain mutations such as deletions, additions, or substitutions. All that is required is that the variant polypeptide have at least 5% (e.g., 10%, 20%, 30%, 40%, 50%, 60%, 70%, 80%, 90%, 95%, 99⁰ 0, 100%, or even more) of the ability of the polypeptide containing only wild-type sequences to specifically function. Substitutions will preferably be conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Variant polypeptides, e.g., those having one or more amino acid substitutions relative to a native polypeptide amino acid sequence, can be prepared and modified as described herein. An artisan in the field would be familiar with the techniques for preparing such polypeptide variants.

In certain embodiments, the protein comprises the amino-acid sequence set forth in SEQ ID NO: 1. In certain embodiments, the protein comprises the amino-acid sequence set forth in SEQ ID NO: 2. In certain embodiments, the protein comprises the amino-acid sequence set forth in SEQ ID NO: 3. In certain embodiments, the protein comprises the amino-acid sequence set forth in SEQ ID NO 4. In certain embodiments, the protein comprises the amino-acid sequence set forth in SEQ ID NO: 5. In certain embodiments, the protein comprises the amino-acid sequence set forth in SEQ ID NO: 6.

In certain embodiments, the protein comprises an amino acid sequence that is at least about 70%, at least about 75%, at least about 80%, at least about 85%, at least about 90%, at least about 95%, at least about 96%, at least about 97%, at least about 98%, at least about 99% identical to the amino acid sequence of: SEQ ID NO. 1, SEQ ID NO. 2, SEQ ID NO. 3, SEQ ID NO. 4, SEQ ID NO. 5, or SEQ ID NO. 6.

In certain embodiments, the protein comprises an amino acid sequence of Bacterial Pesticidal Protein Resource Center (BPPRC) Accession Number MW238546. In certain embodiments, the protein comprises an amino acid sequence of BPPRC Accession Number MW238542. In certain embodiments, the protein comprises an amino acid sequence of BPPRC Accession Number MW238547. In certain embodiments, the protein comprises an amino acid sequence of BPPRC Accession Number MW238548. In certain embodiments, the protein comprises an amino acid sequence of BPPRC Accession Number MW238549. In certain embodiments, the protein comprises an amino acid sequence of BPPRC Accession Number MW238544. In certain embodiments, the protein comprises an amino acid sequence of BPPRC Accession Number MW238545. In certain embodiments, the protein comprises an amino acid sequence of BPPRC Accession Number MW238541.

The present invention further provides, in another aspect, a mixture of proteins, comprising at least one protein comprising the amino-acid sequence set forth in any one of SEQ ID NOs: 1-6.

A person of the art would understand that such a protein mixture includes, but is not limited to, a single protein which comprises a single amino-acid sequence of the indicated amino-acid sequences and another non-indicated protein, a single protein which comprises a single amin oacid sequence of the indicated amino-acid sequences and another single protein which comprises a single amino-acid sequence of the indicated amino-acid sequences, and pluralities thereof.

In certain embodiments, the protein mixture comprises a protein mixture of the M98 strain. In certain embodiments, the protein mixture comprises a protein extract of M98 strain. In certain embodiments, the M98 strain protein mixture comprises proteins comprising the amino-acid sequences set forth in Table 1. In certain embodiments, the protein mixture comprises proteins comprising the amino-acid sequences set forth in SEQ ID NOs: 1-5, Chitinase C, and Chitinase.

In certain embodiments, the protein mixture comprises a protein mixture of the M 100 strain. In certain embodiments, the protein mixture comprises a protein extract of M100 strain. In certain embodiments, the M100 strain protein mixture comprises proteins comprising the amino acid sequences set forth in Table 1. In certain embodiments, the protein mixture comprises proteins comprising the amino-acid sequences set forth in Cry 11a10, Cry2Ab 1, Cry9Eal, SEQ ID NO: 6, Chitinase, and Endochitinase.

In certain embodiments, the protein mixture comprises two or more proteins described herein in detail, including 2, 3, 4, 5, 6 or more proteins. In certain embodiments, the protein mixture comprises at least 2 proteins. In certain embodiments, the protein mixture comprises 2 different proteins. In certain embodiments, the protein mixture comprises 3 different proteins. In certain embodiments, the protein mixture comprises 4 different proteins. In certain embodiments, the protein mixture comprises 5 different proteins. In certain embodiments, the protein mixture comprises 6 different proteins.

In certain embodiments, the protein mixture is a pesticide composition. In certain embodiments, the pesticide composition comprises at least 2 proteins selected from: CrylIa10, Cry2Ab1, Cry9Ea1, and the amino-acid sequence set forth in SEQ ID NO: 6 (M100 Vip3Aa79), or any combination thereof. In certain embodiments, at least 2 proteins are 2, 3, or 4 proteins selected from the group consisting of: CrylIa10, Cry2Ab1, Cry9Ea1, and the amino-acid sequence set forth in SEQ ID NO: 6. Each possibility represents a separate embodiment of the present invention. In certain embodiments, the pesticide composition comprises CrylIa10, Cry2Ab1, Cry9Ea1, and the amino-acid sequence set forth in SEQ ID NO: 6.

In certain embodiments, the pesticide composition comprises: (a) at least 2 proteins selected from: CrylIa10, Cry2Ab1, Cry9Ea1, and the amino-acid sequence set forth in SEQ ID NO: 6; and, (b) at least one protein selected from the group consisting of: Chitinase, and Endochitinase. In certain embodiments, the pesticide composition comprises CrylIa10, Cry2Ab1, Cry9Ea1, the amino-acid sequence set forth in SEQ ID NO: 6, and at least one protein selected from the group consisting of: Chitinase, and Endochitinase. In certain embodiments, the pesticide composition comprises CrylIa10, Cry2Ab1, Cry9Ea1, the amino-acid sequence set forth in SEQ ID NO: 6, Chitinase, and Endochitinase.

The present invention further provides, in another aspect, a nucleic-acid construct, comprising a nucleic-acid sequence encoding the protein described above, or the protein mixture described above.

A person of the art would understand that due to DNA codon degeneracy, multiple, different nucleic-acid constructs, comprising different nucleic-acid sequences, can encode the protein described above, or the protein mixture described above.

In certain embodiments, the nucleic-acid construct comprises a nucleic-acid sequence encoding the protein described above. In certain embodiments, the nucleic-acid construct comprises a nucleic-acid sequence encoding the protein mixture described above.

A person of the art would understand that the nucleic-acid construct includes, but is not limited to, a single nucleic-acid construct encoding a single protein described above, a single nucleic-acid construct encoding different proteins described above, a single nucleic-acid construct encoding a protein mixture described above, different nucleic-acid constructs encoding different protein mixtures described above, and pluralities thereof.

As used herein, the terms “nucleic acid” and “polynucleotide” are used interchangeably to refer to both RNA and DNA, including DNA, genomic DNA, synthetic DNA, and DNA (or RNA) containing nucleic acid analogs, any of which may encode a polypeptide or protein disclosed herein. Polynucleotides can have essentially any three-dimensional structure. A nucleic acid can be double-stranded or single-stranded (i.e., a sense strand or an antisense strand).

The present invention further provides, in another aspect, a nucleic-acid construct, comprising the nucleic-acid sequence set forth in any one of SEQ ID NO 7 (M98 Cry IBkl), SEQ ID NO. 8 (M98 Cryi1a43), SEQ ID NO. 9 (M98 Cry2AbX), SEQ ID NO. 10 (M98 Vip3Aa80), SEQ ID NO: 11 (M98 Vpb1Ac2), SEQ ID NO: 12 (MIOO Vip3Aa79).

A person of the art would understand that such a nucleic-acid construct includes, but is not limited to, a single nucleic-acid construct which comprises a single nucleic-acid sequence of the indicated nucleic-acid sequences, a single nucleic-acid construct which comprises different indicated nucleic-acid sequences, different nucleic-acid constructs which comprise different indicated nucleic-acid sequences, and pluralities thereof.

In certain embodiments, the nucleic-acid construct comprises the nucleic-acid sequence set forth in SEQ ID NO: 7. In certain embodiments, the nucleic-acid construct comprises the nucleicacid sequence set forth in SEQ ID NO: 8. In certain embodiments, the nucleic-acid construct comprises the nucleic-acid sequence set forth in SEQ ID NO: 9. In certain embodiments, the nucleic-acid construct comprises the nucleic-acid sequence set forth in SEQ ID NO: 10. In certain embodiments, the nucleic-acid construct comprises the nucleic-acid sequence set forth in SEQ ID NO 11. In certain embodiments, the nucleic-acid construct comprises the nucleic-acid sequence set forth in SEQ ID NO: 12.

Nucleic acids constructs, that is, nucleic acids having a nucleotide sequence of any of the sequences disclosed herein, can include nucleic acids sequences that are at least about 70%, at least about 75%, at least about 80%, at least about 85%, at least about 90%, at least about 95%, at least about 96%, at least about 97%, at least about 98%, at least about 99% identical to the nucleic acid sequence of: SEQ ID NO. 7, SEQ ID NO. 8, SEQ ID NO. 9, SEQ ID NO. 10, SEQ ID NO. 11, or SEQ ID NO. 12.

The present invention further provides, in another aspect, a mixture of nucleic-acid constructs, comprising at least one nucleic-acid construct comprising the nucleic-acid sequence set forth in any one of SEQ ID NOs: 7-12.

A person of the art would understand that such a nucleic-acid construct mixture includes, but is not limited to, a single nucleic-acid construct which comprises a single nucleic-acid sequence of the indicated nucleic-acid sequences and another non-indicated nucleic-acid, a single nucleic acid construct which comprises a single nucleic-acid sequence of the indicated nucleic-acid sequences and another single nucleic-acid construct which comprises a single nucleic-acid sequence of the indicated nucleic-acid sequences, and pluralities thereof.

In certain embodiments, the mixture of nucleic-acid constructs comprises a nucleic-acid construct comprising the nucleic-acid sequence set forth in SEQ ID NO: 7. In certain embodiments, the mixture of nucleic-acid constructs comprises a nucleic-acid construct comprising the nucleic-acid sequence set forth in SEQ ID NO: 8. In certain embodiments, the mixture of nucleic-acid constructs comprises a nucleic-acid construct comprising the nucleic-acid sequence set forth in SEQ ID NO: 9. In certain embodiments, the mixture of nucleic-acid constructs comprises a nucleic-acid construct comprising the nucleic-acid sequence set forth in SEQ ID NO: 10. In certain embodiments, the mixture of nucleic-acid constructs comprises a nucleic-acid construct comprising the nucleic-acid sequence set forth in SEQ ID NO: 11. In certain embodiments, the mixture of nucleic-acid constructs comprises a nucleic-acid construct comprising the nucleic-acid sequence set forth in SEQ ID NO: 12.

In certain embodiments, the mixture of nucleic-acid constructs comprises a nucleic-acid construct comprising the nucleic-acid sequences set forth in SEQ ID NOs: 7-11, a nucleic-acid sequence encoding Chitinase C, and a nucleic-acid sequence encoding Chitinase.

In certain embodiments, the mixture of nucleic-acid constructs comprises a nucleic-acid construct comprising the nucleic-acid sequences set forth in SEQ ID NO: 7, a nucleic-acid construct comprising the nucleic-acid sequences set forth in SEQ ID NO: 8, a nucleic-acid construct comprising the nucleic-acid sequences set forth in SEQ ID NO: 9, a nucleic-acid construct comprising the nucleic-acid sequences set forth in SEQ ID NO: 10, a nucleic-acid construct comprising the nucleic-acid sequences set forth in SEQ ID NO: 11, a nucleic-acid sequence encoding Chitinase C, and a nucleic-acid sequence encoding Chitinase.

In certain embodiments, the mixture of nucleic-acid constructs comprises a nucleic-acid construct comprising a nucleic-acid sequence encoding CrylIa10, a nucleic-acid sequence encoding Cry2Abl, a nucleic-acid sequence encoding Cry9Eal, the nucleic-acid sequence set forth in SEQ ID NO: 12, a nucleic-acid sequence encoding Chitinase, and a nucleic-acid sequence encoding Endochitinase.

In certain embodiments, the mixture of nucleic-acid constructs comprises two or more nucleic-acid constructs described herein in detail, including 2, 3, 4, 5, 6 or more nucleic-acid constructs. In certain embodiments, the mixture of nucleic-acid constructs comprises at least 2 nucleic-acid constructs. In certain embodiments, the mixture of nucleic-acid constructs comprises 2 different nucleic-acid constructs. In certain embodiments, the mixture of nucleic-acid constructs comprises 3 different nucleic-acid constructs. In certain embodiments, the mixture of nucleic-acid constructs comprises 4 different nucleic-acid constructs. In certain embodiments, the mixture of nucleic-acid constructs comprises 5 different nucleic-acid constructs. In certain embodiments, the mixture of nucleic-acid constructs comprises 6 different nucleic-acid constructs.

In certain embodiments, the present invention provides a composition comprising a vector comprising at least one nucleic-acid construct comprising the nucleic-acid sequence set forth in any one of SEQ ID NOs: 7-12.

In certain embodiments, the present invention provides a composition comprising a vector comprising at least one recombinant polynucleotide encoding the amino-acid sequence set forth in any one of SEQ ID NOs: 1-6.

In certain embodiments, the present invention provides at least one nucleic-acid construct comprising at least one nucleic-acid sequence encoding at least two proteins selected from the group consisting of: CrylIa10, Cry2Ab 1, Cry9Ea1, and the amino-acid sequence set forth in SEQ ID NO: 6.

In certain embodiments, the present invention provides at least one nucleic-acid construct comprising a nucleic-acid sequence encoding CrylIa10, a nucleic-acid sequence encoding CrylIa10, a nucleic-acid sequence encoding Cry2Ab1, a nucleic-acid sequence encoding Cry9Ea1, and a nucleic-acid sequence encoding the amino-acid sequence set forth in SEQ ID NO: 6. In certain embodiments, the present invention provides at least one nucleic-acid construct comprising a nucleic-acid sequence encoding CrylIa10, a nucleic-acid sequence encoding CrylIa10, a nucleic-acid sequence encoding Cry2Ab1, a nucleic-acid sequence encoding Cry9Ea1, and the nucleic-acid sequence set forth in SEQ ID NO: 12.

In certain embodiments, the present invention provides at least one nucleic-acid construct comprising at least one nucleic-acid sequence encoding: (a) at least two proteins selected from the group consisting of: CrylIa10, Cry2Ab1, Cry9Ea1, and the amino-acid sequence set forth in SEQ ID NO: 6; and, (b) at least one protein selected from: Chitinase, Endochitinase, and both. In certain embodiments, the present invention provides at least one nucleic-acid construct comprising a nucleic-acid sequence encoding CrylIa10, a nucleic-acid sequence encoding CrylIa10, a nucleic-acid sequence encoding Cry2Ab1, a nucleic-acid sequence encoding Cry9Ea1, the nucleic-acid sequence set forth in SEQ ID NO: 12, and at least one protein selected from: Chitinase, Endochitinase, and both. In certain embodiments, the present invention provides at least one nucleic-acid construct comprising a nucleic-acid sequence encoding CrylIa10, a nucleic-acid sequence encoding Cry1Ia10, a nucleic-acid sequence encoding Cry2Ab1, a nucleic-acid sequence encoding Cry9Ea1, the nucleic-acid sequence set forth in SEQ ID NO: 12, a nucleic-acid sequence encoding Chitinase, and a nucleic-acid sequence encoding Endochitinase.

In certain embodiments, at least one nucleic acid construct is 1, 2, 3, 4, 5, or 6 nucleic acid constructs. Each possibility represents a separate embodiment of the present invention.

As used herein the term “recombinant polynucleotide” refers to a polynucleotide having a genetically engineered modification introduced through manipulation via mutagenesis, restriction enzymes, and the like. Recombinant polynucleotides may comprise DNA segments obtained from different sources, or DNA segments obtained from the same source, but which have been manipulated to join DNA segments which do not naturally exist. A recombinant polynucleotide may exist outside of the cell, for example as a PCR fragment, or integrated into a genome, such as a bacteria or plant genome.

In one embodiment, a polynucleotide of the present invention (nucleic-acid construct) is operatively linked in a recombinant polynucleotide to a promoter functional in a plant or bacteria to provide for expression of the polynucleotide in the sense orientation such that a desired polypeptide is produced. Also considered are embodiments wherein a polynucleotide is operatively linked to a promoter functional in a plant to provide for expression of the polynucleotide in the antisense orientation such that a complimentary copy of at least a portion of an mRNA native to the target plant host is produced. Such a transcript may contain both sense and antisense regions of a polynucleotide, for example where RNAi methods are used for gene suppression.

In one embodiment, the promoter of the expression vector of the present invention is operably linked to the polynucleotide (nucleic-acid construct). In one embodiment, the promoter is a constitutive promoter. In another embodiment, the promoter is an inducible promoter. In another embodiment, the promoter is a tissue-specific promoter. In another embodiment, the promoter is an organ-specific promoter. In one embodiment, a promoter used in the compositions and methods of the present invention is cisgenic, i.e. is a promoter that is native to the plant.

Recombinant polynucleotides of the present invention are assembled in recombinant DNA constructs using methods known to those of ordinary skill in the art. Thus, DNA constructs used for transforming plant cells will comprise a polynucleotide one desires to introduce into a target plant. Such constructs will also typically comprise a promoter operatively linked to said polynucleotide to provide for expression in the target plant. Other construct components may include additional regulatory elements, such as 5′ or 3′ untranslated regions (such as polyadenylation sites), intron regions, and transit or signal peptides. Furthermore, the promoters may be altered to contain multiple “enhancer sequences” to assist in elevating gene expression. Such enhancers are known in the art.

The present invention contemplates the use of polynucleotides (or nucleic-acid constructs) effective for imparting an enhanced phenotype to genetically modified plants or bacteria expressing said polynucleotides or nucleic-acid constructs.

The present invention further provides, in another aspect, a cell, comprising the protein described above, the protein mixture described above, the nucleic-acid construct described above, the nucleic-acid construct mixture described above, or any combination thereof

In certain embodiments, the cell comprises the protein described above. In certain embodiments, the cell comprises the protein mixture described above. In certain embodiments, the cell comprises the nucleic-acid construct described above. In certain embodiments, the cell comprises the nucleic-acid construct mixture described above.

In certain embodiments, the cell is a eukaryote cell. In certain embodiments, the cell is a yeast cell. In certain embodiments, the cell is a prokaryote cell. In certain embodiments, the cell is a bacterium cell. In certain embodiments, the cell is a Bacillus thuringiensis bacterium cell. In certain embodiments, the cell is a plant cell.

Provided herein are host cells comprising a vector, e.g., a DNA plasmid which supports the replication and/or expression of the vector. Host cells may be prokaryotic cells such as E. coli, or eukaryotic cells such as yeast, plant, insect, amphibian, or mammalian cells. In certain embodiments, host cells are monocotyledonous or dicotyledonous plant cells. In certain embodiments, the host cell utilized in the methods of the present invention is transiently transfected with the nucleic acid construct described herein in detail.

In certain embodiments, the introduced nucleotide sequence (nucleic-acid construct) is incorporated into a plasmid or vector capable of autonomous replication in a cell. Any of a wide variety of vectors can be employed for this purpose and are known and available to those of ordinary skill in the art. The most suitable plasmid or vector is selected based on the ability to select cells that contain the vector from those cells which do not contain the vector; the number of copies of the vector which are desired in a particular host cell; and whether it is desirable to be able to “shuttle” the vector between host cells of different species. In one embodiment, the nucleic acid construct is integrated into the plant or bacteria chromosome. In another embodiment, the nucleic-acid construct is expressed from a vector.

In certain embodiments, the cell comprising the protein described above, the protein mixture described above, the nucleic-acid construct described above, the nucleic-acid construct mixture described above, or any combination thereof, may be used for controlling pests. In certain embodiments, the protein described above, the protein mixture described above, the nucleic-acid construct described above, or the nucleic-acid construct mixture described above may be used for controlling pests. In certain embodiments, the cell described above, the protein described above, the protein mixture described above, the nucleic-acid construct described above, or the nucleic acid construct mixture described above may be used as an insecticide.

In certain embodiments disclosed herein is an insecticidal composition comprising the cell described above, the protein described above, the protein mixture described above, the nucleic acid construct described above, the nucleic-acid construct mixture described above, or any combination thereof.

As used herein, the terms “insecticidal”, “insecticide” and “pesticide” are interchangeable, and refer to the ability of an agent to increase insect or pest mortality.

In certain embodiments, the pesticide composition disclosed herein comprises a bacterial cell or a portion thereof.

As used herein, the term “portion thereof” refers to any portion of the bacterial cell, or any metabolic state in which the bacterial cell is present. Non-limiting examples include: membranal proteins, endogenous proteins, secreted proteins (or secretome), nucleic acids, and a bacterial spore. In certain embodiments, a portion of a bacterial cell comprises an endospore. In certain embodiments, a portion of the bacterial cell comprises a crystal protein. In certain embodiments, a portion of the bacterial cell comprises several crystal proteins. In certain embodiments, a portion of the bacterial cell comprises a complex comprising an endospore and a crystal protein.

A person having ordinary skill in the art would be familiar with methods for production of the composition disclosed herein. In certain embodiments, the composition is produced according to standard protocols for producing BTK standard product, BTA standard product, and both. In certain embodiments, the bacterial cell used for the pesticide composition is grown in a sterile media comprising carbon, nitrogen and trace minerals. In certain embodiments, inoculum amounts of the bacterial cell range from 0.05 to 5% of the fermenter volume. In certain embodiments, after approximately two days of vigorous vegetative growth, the bacterial cell begins to sporulate in response to reduced available nutrients, forming at one end of the cell a dormant endospore and at the other end the protein crystal, which contains the toxins. In certain embodiments, once sporulation has been completed the media is treated to destroy vegetative cells. The spore-crystal complex can be exposed to temperatures of 180° C. for short periods of time without degradation. In certain embodiments, endospores and crystals are concentrated from the fermentation broth by either centrifugation or filtration. In certain embodiments, the resultant spores and crystals are spray dried to form a fine technical powder, and later granulated to form Water Dispersible Granules (WDGs).

In certain embodiments, the bacterial cell belongs to Bacillus Thuringiensis specie. In certain embodiments, the bacterial cell is Bacillus Thuringiensis M100 strain.

In certain embodiments the insecticidal composition is in the form of an aqueous suspension, an oil suspension, a dry or a wettable granule, powder, dust, pellet, or colloidal dispension. In certain embodiments, the insecticidal composition further comprises a carrier, stabilizer, additive, surfactant, adjuvant, emulsifier, dispersant, or any material suitable for agricultural application. Such carriers, stabilizers, additives, surfactants, adjuvants, emulsifiers, dispersants, or materials suitable for agricultural application can be solid or liquid and are well known in the art. In certain embodiments, the insecticidal composition further comprises other insecticides, pesticides, or active agents.

In certain embodiments, the amount of the insecticidal composition or the agent of the invention is applied at an insecticidally-effective amount. A skilled artisan would be familiar with methods of determining the insecticidally-effective amount, which depends on factors such as, the specific target pest, the specific plant or crop to be treated, the environmental conditions, the application method, and concentration of the insecticidal composition or the agent of the invention. In certain embodiments, the insecticidal composition or agent of the invention is applied to a particular pest, plant or target area in one or more applications, as needed.

The present invention further provides, in another aspect, a method of controlling a pest, comprising contacting the pest with the protein described above, the protein mixture described above, the nucleic-acid construct described above, the nucleic-acid construct mixture described above, the cell described above, or any combination thereof.

A person of the art would understand that the phrase “controlling a pest” as used herein includes, but is not limited to, preventing a pest from living, preventing a pest from causing agricultural damage, preventing pest replication, attenuating pest activity, and killing pest.

A person of the art would understand that the term “contacting” as used herein generally refers to bringing the agent of the invention or insecticidal composition described herein to conditions, e.g. sufficient proximity, such that the agent can interact with the pest. In certain embodiments, contacting comprises topical and/or systemic application to field crops, grasses, fruits, vegetables, and plants. In certain embodiments, the agent of the invention comprises the cell described above, the protein described above, the protein mixture described above, the nucleicacid construct described above, the nucleic-acid construct mixture described above, or any combination thereof

In certain embodiments, the agent of the invention or insecticidal composition described herein is applied to the environment of the pest (or target insect). In certain embodiments, the agent of the invention or insecticidal composition described herein is applied to the foliage of the plant or crop. In certain embodiments, the agent of the invention or insecticidal composition described herein is externally applied to a plant, or to the environment surrounding the plant. In certain embodiments, the agent of the invention or insecticidal composition described herein is applied to pest food to be consumed by the pest. In certain embodiments, the agent of the invention or insecticidal composition described herein is applied to a forest area. In certain embodiments, the agent of the invention or insecticidal composition described herein is applied to a tree.

In certain embodiments, the agent of the invention is applied by conventional methods, including spraying, dusting, sprinkling, soaking, soil injection, seed coating, seedling coating, spraying, aerating, misting, atomizing, and the like, which are well-known to those of skill in the art. In certain embodiments, the pest is killed by ingestion of the agent of the invention or insecticidal composition described herein.

In certain embodiments, the method comprises contacting the pest with the protein described above. In certain embodiments, the method comprises contacting the pest with the protein mixture described above. In certain embodiments, the method comprises contacting the pest with the nucleic-acid construct described above. In certain embodiments, the method comprises contacting the pest with the nucleic-acid construct mixture described above. In certain embodiments, the method comprises contacting the pest with the cell described above. In certain embodiments, the method comprises contacting the pest with the insecticidal composition described above.

In certain embodiments, the method comprises contacting the pest with an effective amount of the composition disclosed herein. In certain embodiments, the effective amount is determined according to international toxic unit (ITU) / mg. In certain embodiments, the effective amount is in the range of 100- 200,000 ITU/mg. In certain embodiments, the effective amount is in the range of 100 - 200,000 ITU/mg, 200 — 200,000 ITU/mg, 300 — 200,000 ITU/mg, 400 — 200,000 ITU/mg, or 500 — 200,000 ITU/mg. Each possibility represents separate embodiment of the present invention. In certain embodiments, the effective amount is in the range of 500 - 190,000 ITU/mg, 500 — 180,000 ITU/mg, 500 — 170,000 ITU/mg, or 500 — 160,000 ITU/mg. Each possibility represents separate embodiment of the present invention. In certain embodiments, the effective amount is in the range of 150- 160,000 ITU/mg.

In certain embodiments, contacting is for less than 4 days. In certain embodiments, contacting is for 1-4, 1-3, or 1-2 days. Each possibility represents a separate embodiment of the present invention. In certain embodiment, contacting is for 6 hr — 96 hr, 6 hr — 72 hr, 6 hr — 48 hr, 6 hr - 24 hr, 12 hr - 96 hr, 12 hr - 72 hr, 12 hr —48 hr, 12 hr - 24 hr, 18 hr - 96 hr,, 18 hr - 72 hr, 18 hr — 48 hr, 18 hr — 24 hr, 24 hr — 96 hr, 24 hr -72 hr, 24 hr — 48 hr, 30 hr -96 hr, 30 hr - 72 hr, 30 hr - 48 hr, 36 hr — 96 hr, 36 hr — 72 hr, 36 hr — 48 hr, 42 hr — 96 hr, 42 hr — 72 hr, 42 hr — 48 hr, 48 hr — 96 hr, 48 hr — 72 hr, 54 hr -96 hr, 54 hr — 72 hr, 60 hr — 96 hr, 60 hr — 72 hr, 66 hr — 96 hr, 66 hr — 72 hr, 72 hr — 96 hr, 78 hr — 96 hr, 84 hr — 96 hr, or 90 hr — 96 hr. Each possibility represents a separate embodiment of the present invention.

In certain embodiments, the pest is an insect pest. In certain embodiments, the pest is a moth. In certain embodiments, the pest is a beetle.

In certain embodiments, the pest is of the Phylum Arthropoda. In certain embodiments, the pest is of the Class Insecta. In certain embodiments, the pest is of the Order Lepidoptera. In certain embodiments, the pest is of the Family Tortricidae. In certain embodiments, the pest is of the Genus Thaumatotibia. In certain embodiments, the pest is of the Subgenus Thaumatotibia (Cryptophlebia). In certain embodiments, the pest is of the Species T. leucoireta. In certain embodiments, the pest is of the Family Plutellidae. In certain embodiments, the pest is of the Genus Plutella. In certain embodiments, the pest is of the Species P. xylostella.

In certain embodiments, the pest is of the Superfamily Pyraloidea. In certain embodiments, the pest is of the Family Pyralidae.

In certain embodiments, the pest is of the Order Coleoptera. In certain embodiments, the pest is of the Suborder Polyphaga. In certain embodiments, the pest is of the Infraorder Cucujiformia. In certain embodiments, the pest is of the Superfamily Tenebrionoidea. In certain embodiments, the pest is of the Family Tenebrionidae.

In certain embodiments, the pest is of the Superfamily Noctuoidea. In certain embodiments, the pest is of the Family Noctuidae. In certain embodiments, the pest is of the Genus Spodoptera. In certain embodiments, the pest is of the Species S. littoralis. In certain embodiments, the pest is of the Species S. Frugiperda. In certain embodiments, the pest is of the Species S. eridania.

In certain embodiments, the pest is of the Family Thaumetopoeidae. In certain embodiments, the pest is of the Genus Thaumetopoea. In certain embodiments, the pest is of the Species T. pityocampa.

In certain embodiments, the pest is of the Superfamily Noctuoidea. In certain embodiments, the pest is of the Family Notodontidae. In certain embodiments, the pest is of the Genus Thaumetopoea. In certain embodiments, the pest is of the Subfamily Heliothinae. In certain embodiments, the pest is of the Genus Chloridea. In certain embodiments, the pest is of the Species C. virescenes.

In certain embodiments, the Carob moth is Ectomyelois ceratoniae. In certain embodiments, the Carob moth is Cadra calidella.

In certain embodiments, the pest is a lepidopteran pest. In certain embodiments, the pest is a lepidopteran pest larva.

In certain embodiments, the pest is of the family Gelechiidaet. In certain embodiments, the pest is of the Genus Tuta. In certain embodiments the pest is of the Species T. absoluta.

In certain embodiments, the pest is selected from the group consisting of False codling moth, Carob moth, Darkling beetle, Spodoptera litorallis, Pine processionary moth, and Pistachio processionary moth, Spodoptera Frugiperda, Spodoptera eridania, Heliothis virescens, Plutella xylostella, and Tuta absoluta.

In certain embodiments, the pest is False codling moth. In certain embodiments, the pest is Carob moth. In certain embodiments, the pest is Darkling beetle. In certain embodiments, the pest is Spodoptera litorallis. In certain embodiments, the pest is Pine processionary moth. In certain embodiments, the pest is Pistachio processionary moth. In certain embodiments, the pest is Spodoptera frugiperda. In certain embodiments, the pest is a fall armyworm. In certain embodiments, the pest is Spodoptera eridania. In certain embodiments the pest is a southern armyworm. In certain embodiments, the pest is Heliothis virescens (or Chloridea virescens. In certain embodiments, the pest is tobacco budworm. In certain embodiments, the pest is Plutella xylostella. In certain embodiments, the pest is diamondback moth. In certain embodiments, the pest is Tuta absoluta.

In certain embodiments, the protein described above is synthetic. In certain embodiments, the protein mixture described above is synthetic. In certain embodiments, the nucleic-acid construct described above is synthetic. In certain embodiments, the nucleic-acid construct mixture described above is synthetic. In certain embodiments, cell described above is synthetic.

A person of the art would understand that the term “synthetic” as used herein generally means non-natural, not found in nature, or man-made. It should be understood that a composition is considered “synthetic” if it is not found as a whole as-is in nature.

In certain embodiments, the protein described above is isolated. In certain embodiments, the protein mixture described above is isolated. In certain embodiments, the nucleic-acid construct described above is isolated. In certain embodiments, the nucleic-acid construct mixture described above is isolated. In certain embodiments, cell described above is isolated.

A person of the art would understand that the term “isolated” as used herein generally means not found in its natural surroundings. It should be understood that a composition is considered “isolated” if it is purified, e.g. with at least 95% purity, or if it is mixed with other compositions which are not found in its natural surroundings. A chemically synthesized nucleic acid or polypeptide or one synthesized using in vitro transcription/translation is considered “isolated.”

Isolated nucleic acid molecules can be produced by in several ways. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

The nucleic acids and polypeptides described herein may be referred to as “exogenous”. The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

In certain embodiments, the protein described above is encoded by a codon-optimized nucleic-acid sequence. In certain embodiments, the protein mixture described above is encoded by a codon-optimized nucleic-acid sequence. In certain embodiments, the nucleic-acid construct described above comprises a codon-optimized nucleic-acid sequence. In certain embodiments, the nucleic-acid construct mixture described above comprises a codon-optimized nucleic-acid sequence. In certain embodiments, cell described above comprises a codon-optimized nucleic-acid sequence or a protein encoded by a codon-optimized nucleic-acid sequence.

A person of the art would understand that different cells exhibit bias towards use of certain codons over others for the same amino acid, and that this bias can significantly impact expression. In certain embodiments, nucleic-acid sequences described above are codon-optimized according to the cell described above in which they encode the proteins described above.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### EXAMPLES

### Example 1

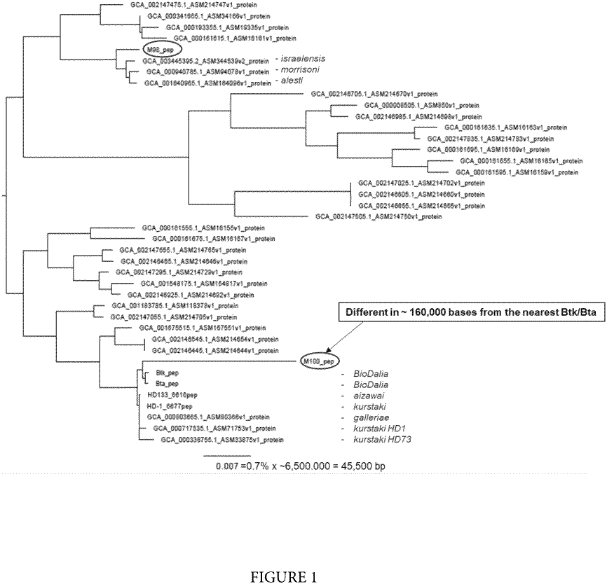

Bacillus thuringiensis (Bt) cells were isolated from soil samples and insect cadavers, enriched from the isolates by growth in Luria Bertani (LB) medium containing 0.25 M acetate, which selectively inhibits germination of their spores and not that of other spore-formers, and plated on LB agar following a heat shock. Single colonies were grown in liquid T3 medium and selected for the appearance of parasporal inclusions by phase-contrast microscopy. Samples (after 96 hours of growth) were frozen at -70° C. with 15% glycerol or lyophilized after being washed with sterile distilled water by centrifugation. New strains were identified as comprising unique combinations of anti-pest toxin genes. FIG. 1 shows the phylogenetic relationships (unrooted tree) of 40 Bacillus thuringiensis genomic sequences, including the newly-identified isolates M98 and M100. The M100 strain is different by about 160,000 bases from the nearest BtWBti strain. Table 1 summarizes the toxic-type protein profiles of known reference strains (aizawai HD-133 and kurstaki HD- 1), as well as of M98 and M100 strains that encode newly-identified toxins.

TABLE 1

| | | | | | |
|---|---|---|---|---|---|
| B. thuringiensis strains | Cryl | Cry2 | Cry9 | Vip / vpb | Chitinase producer |
| aizawai HD-133 (BTA) | Cry1Aa, Cry1Ab, Cry1Ca, Cry1Da, Cry1I | Cry2Ab | Cry9 | Vip | Yes |
| kurstaki HD-I (BTK) | Cry1Aa, Cry1Ab, Cry1Ac, Cry1I | Cry2Aa, Cry2Ab | | Vip3Aa | Yes |
| M98 | Crv1Bk1 | Crv2AbX | | Vip3Aa80 | Chitinase C, Chitinase |
| | (88.0% identity to Cry1Bal/ CrylBa5) Crv11a43 (96.0% identity to Cry llal 0/C11a14 | (92.7% identity to Cry2Ab 1) | | (99.2% identity to Vip3Aa10/Vip3Aa11) Vpb1Ac2 (99.5% identity to Vpb1Ac1) | |
| M100 (BTM) | Cry1Ia42 (100% identity to Cry1la10) | Cry2Ab41 (100% identity to Cry2Ab 1) | Cry9Ea12 (100% identity to Cry9Ea1 | Vip3Aa79 (99.7% identity to Vip3Aa10/Vip3Aa11) | Chitinase, Endochitinase |

As shown in Table 1, newly-identified strains M98 and M 100 not only encode newlyidentified toxins, herein referred to as Cry1Bk1, Cry11a43, Cry2AbX, Vip3Aa80, VpblAc2, and Vip3Aa79 (bold and underlined), they further comprise new combinations of known and newly identified toxin proteins.

### Example 2

The anti-pest activities of the newly-identified strains M98 and M100 were bioassayed against several key pests.

For Spodoptera litorallis and Alphitobius diaperinus, larvae were measured and equal amount of Bt culture of either of the examined strains were mixed with stone fly Heliothis premix diet. Diet was divided equally to Petri dishes and 10 neonate larvae of each pest were added in each plate and checked for the mortality daily for up to 10 days. Petri dishes were sealed with Parafilm and incubated at 25 ⁰C, with a 12: 12 LD photoperiod.

For pine processionary moth, small glass test tubes (95 × 10 mm, length × internal diameter) each containing three brachyblasts (a total of six needles) and ten larvae, were used. Needles were immersed in the respective Bt culture and left to dry before inserting into the tube. Each tube constituted a replicate and there were five replicates per treatment. The tubes were closed with cling-plastic adhesive wrap and incubated at 23 ⁰C, 60- 80% RH, and with a 12:12 L:D photoperiod.

For Pistachio processionary moth, Pistacia fresh leaves were immersed in the respective Bt culture and left to dry before inserting into Petri dishes. Ten neonate larvae were added in each plate and checked for the mortality daily for up to 10 days. Petri dishes were sealed with Parafilm and incubated at 22⁰C, with a 12:12 LD photoperiod.

Table 2 summarizes the cry genes and toxicities of reference strains (BTK, BTA and BTT) and newly-identified (M98 and M100) B. thuringiensis strains.

TABLE 2

| BT strain | Toxicity Level or LC₅₀ (% of harvested, sporulate culture)* | | | | | |
|---|---|---|---|---|---|---|
| | Thauma totihia leucotret a “False codling moth” | Ectoyelois ceratoniae “Carob moth” | Alphitobius diaperinus “Darkling beetle” | Spodoptera litorallis “African cotton leafworm” | Thaumetopoea pityocampa “Pine processionary moth” | Thaumetopoe a solitaria “Pistachio processionar y moth” |
| BTK (kurstakl) | 0.0016 | Toxic | | | 0.92 High toxicity | 2.26 Toxic |
| BTA (aizawaz) | | | | 0.78 | | 2.99 High toxicity |
| BTT (tenebrionis) | | | No toxicity | | | |
| M100 | 0.0009 High toxicity | 0.012 High toxicity | | 1.04 Moderate toxicity | 1.2 High toxicity | 2.92 High toxicity |
| M98 | 0.0010 High toxicity | 0.498 High toxicity | | Moderate toxicity | 4.38 High toxicity | 3.55 High toxicity |

* Numbers refer to LC50. (-) means “not yet determined”.

As shown in Table 2, M98 and M 100 are moderately toxic against Spodoptera litorallis, and highly toxic to False codling moth, Carob moth, Pine processionary moth, and Pistachio processionary moth.

For examining synergistic activity, the activity of the Bt strains was examined for each newly-identified strain (M98 and M100) separately and for both strains combined or combined with commercial strains. The evaluation was conducted for Spodoptera litorallis and Apomyelois ceratoniae larvae.

The sequences have been deposited with the Bacterial Pesticidal Protein Resource Center (BPPRC) (www.bpprc.org) as shown in Table 3

TABLE 3

| Name | Accession Number | Deposit Year |
|---|---|---|
| Cry I Ia42 | MW238546 | 2020 |
| Cry I Ia43 | MW238542 | 2020 |
| Cry2Ab41 | MW238547 | 2020 |
| Cry9Ea12 | MW238548 | 2020 |
| Vip3Aa79 | MW238549 | 2020 |
| Vip3Aa80 | MW238544 | 2020 |
| Vpb1Ac2 | MW238545 | 2020 |
| Cry I Bk I | MW238541 | 2020 |

### Example 3- Preparation of M100- Based Product; BTM

Spore suspensions were subjected to thermal treatment at a temperature of 75° C. for a duration of 20 minutes. The treated suspensions were then inoculated into flasks containing TSB culture medium. Subsequently, the flasks were subjected to continuous agitation at an optimized growth temperature. Following an appropriate incubation period, the bacterial culture was transferred to a production medium. The growth process was allowed to progress for a duration of 28-44 hours, allowing complete sporulation. A viable cell count was performed to determine the live bacterial population. The culture was then subjected to centrifugation, and the resulting precipitate was combined with formulation materials following protocols that are based on the established protocols for the production of BTK- and BTA-based products. The observed changes in growth parameters, such as oxygen consumption rate, pH variation, and other related factors, were consistent with the typical growth behavior exhibited by BTK and BTA bacteria during fermentation. A bacterial count of 29.2×10 counts/ml was achieved, falling within the range typically obtained at the conclusion of commercial cultivation of these bacteria.

Table 4 describes the toxins content of M100-based product (BTM) compared to BTK and BTA standards.

TABLE 4

| | | | | |
|---|---|---|---|---|
| B. thuringiensis strains | Cry1 | Cry2 | Cry9 | Vip / Vpb |
| BTM margalitus (M100) | Cry1Ia42 (100% identity to Cry1Ia10) | Cry2Ab41 (100% identity to Cry2Ab1) | Cry9Ea12 (100% identity to Cry9Ea1) | Vip3Aa79 (99.7% identity to Vip3Aa10/Vip3aA11) |
| BTK kurstaki | Cry1Aa, Cry1Ab, Cry1Ac Cry1I | Cry2Aa, Cry2Ab | -- | Vip3Aa |
| BTA aizawi | Cry1Aa, Cry1Ab, Cry1Ca, Cry1Da, Cry1I | Cry2Ab | Cry9A | -- |

### Example 4 - Evaluation of M100- Based Product (BTM) Activity Against Spodoptera Eridania

A diet-incorporation bioassay was next performed for potency determination of M100-based product; BTM against Spodoptera eridania larvae, on the product listed below. The B.t.k. internal reference standard used is a technical powder calibrated directly against the international reference standard “HD1-S-1980” using Trichoplusia ni larvae.

TABLE 5

LC50 values (mg/liter) of M100- based product, sample 1

| Replicate | Heterogen. | LC50 | Lower limit | Upper limit | Slope ±SE | Ref Std LC50 | POTENCY |
|---|---|---|---|---|---|---|---|
| 1 | 1.30 | 117.7 | 67.25 | 176.4193 | 2.06 ±0.32 | 457.8 | 311164 |
| 2 | 0.51 | 126.4 | 94.42 | 162.3130 | 1.98 ±0.31 | 487.6 | 308608 |
| 3 | 1.40 | 151.7 | 97.77 | 223.9154 | 2.37 ±0.33 | 450.3 | 237469 |
| Average | | 131.9 | 86.48 | 189.5492 | | 465.23 | 285746 |

As demonstrated in Table 4, the average LC₅₀ value of M100- based product (BTM), sample 1, against Spodoptera eridania was 131.9 mg/liter compared to the BTK standard exhibiting a LC₅₀ value of 465.2 mg/liter, and the average potency value was 285746.

TABLE 5

LC50 values (mg/liter) of M100- based product, sample 2

| Replicate | Heterogen. | LC50 | Lower limit | Upper limit | Slope ±SE | Ref Std LC50 | POTENCY |
|---|---|---|---|---|---|---|---|
| 1 | 0.63 | 275.9 | 218.7 | 341.8170 | 2.54 ±0.36 | 457.8 | 132744 |
| 2 | 0.31 | 311.9 | 239.4 | 381.3197 | 3.10 ±0.54 | 482.2 | 123681 |
| 3 | 2.61 | 272.4 | 137.9 | 428.2970 | 3.30 ±0.47 | 450.3 | 132247.3 |
| Av. | | 286.7 | 198.6 | 383.81 | | 463.43 | 129557 |

As may be seen in Table 5, the average LC₅₀ value of M100- based product, sample 2, against Spodoptera eridania was 131.9 mg/liter compared to the BTK standard exhibiting a LC50 value of 465.2 mg/liter, and the average potency value was 129557.

### Example 5 - Evaluation of M100- Based Product; BTM Activity Against Spodoptera Frugiperda

The toxicity of M100- based product (BTM) was next examined against S. frugiperda larvae, applied on corn plants. The newly hatched first instar larvae of S. frugiperda were kept without feeding until the beginning of experiment. 0.3% solution of each product, BTK standard, and M100- based product (BTM) were prepared, as described above, and applied on the corn leaf embedded on an agarose layer within a petri plate. Each petri plate was introduced with 10 larvae of S. frugiperda and covered with a perforated cap. Petri plates were kept in a rectangular box containing a wet paper towel, for humidity maintenance, covered with a lid and incubated at 25° C. The five replicates of each treatment including control were incorporated. The mortality of the larvae was recorded each day for four days. The experiments were repeated three times.

Wald test yielded a p-value of 0.96, thus the three experiments were combined into a single dataset and analyzed as such. The obtained data were subjected to statistical analysis for standard least squares by restricted maximum likelihood (REML) methods using JumpPro. The experimental parameters were estimated to determine the goodness of fit of the model and found to be significant (Bt-1=BTK standard; and Bt-2=BTM). The significant difference between the response of different groups or treatments was analyzed using analysis of variance (ANOVA) or t-test. Tukey HSD test indicated a significant effect of the 3 fixed effects: treatment, days post inoculation (DPI), and treatment×DPI (<0.0001).

Daily mortality of S. frugiperda larvae exposed to BTK standard during 4 days of observation, is presented in Table 6. The total mortality of the larvae, 4 days post inoculation, was in the range of 74%-76%.

TABLE 6

Daily mortality of S. frugiperda larvae exposed to BTK standard.

| Test 1-BTK | T1 | T2 | T3 | T4 | T5 | Average | % Mortality | Stdev | St. Error |
|---|---|---|---|---|---|---|---|---|---|
| Day 1 | 0 | 1 | 0 | 0 | 0 | 0.2 | 2 | 0.447214 | 0.2 |
| Day 2 | 2 | 5 | 3 | 4 | 3 | 3.4 | 34 | 1.140175 | 0.509902 |
| Day 3 | 5 | 7 | 5 | 5 | 4 | 5.2 | 52 | 1.095445 | 0.489898 |
| Day 4 | 8 | 9 | 8 | 7 | 6 | 7.6 | 76 | 1.140175 | 0.509902 |
| Day 1 | 0 | 0 | 0 | 0 | 1 | 0.2 | 2 | 0.447214 | 0.2 |
| Day 2 | 1 | 0 | 0 | 0 | 2 | 0.6 | 6 | 0.894427 | 0.4 |
| Day 3 | 5 | 7 | 1 | 0 | 6 | 3.8 | 38 | 3.114482 | 1.392839 |
| Day 4 | 8 | 8 | 6 | 6 | 10 | 7.6 | 76 | 1.67332 | 0.748331 |
| Day 1 | 0 | 1 | 0 | 0 | 0 | 0.2 | 2 | 0.447214 | 0.2 |
| Day 2 | 0 | 1 | 3 | 0 | 2 | 1.2 | 12 | 1.30384 | 0.583095 |
| Day 3 | 2 | 1 | 5 | 2 | 3 | 2.6 | 26 | 1.516575 | 0.678233 |
| Day 4 | 6 | 7 | 7 | 7 | 10 | 7.4 | 74 | 1.516575 | 0.678233 |

Daily mortality of S. frugiperda larvae exposed to M100- based product (BTM) during 4 days of observation, is presented in Table 7. The S. frugiperda larvae showed absolute mortality (100%) after 4 days of observation. BTM was observed to be more repellent or deterrent to S. frugiperda larvae, compared to BTK, as the larvae were found to escape the leaf surface, as well as to avoid nibbling the leaves applied with BTM.

TABLE 7

Daily mortality of S. frugiperda larvae exposed to M100- based product (BTM).

| Test 1-BTM | T1 | T2 | T3 | T4 | T5 | Average | % Mortality | Stdev | St. Error |
|---|---|---|---|---|---|---|---|---|---|
| Day 1 | 0 | 0 | 1 | 0 | 0 | 0.2 | 2 | 0.447214 | 0.2 |
| Day 2 | 4 | 3 | 4 | 4 | 5 | 4 | 40 | 0.707107 | 0.316228 |
| Day 3 | 7 | 8 | 10 | 9 | 10 | 8.8 | 88 | 1.30384 | 0.583095 |
| Day 4 | 10 | 10 | 10 | 10 | 10 | 10 | 100 | 0 | 0 |
| Day 1 | 0 | 1 | 1 | 0 | 0 | 0.4 | 4 | 0.547723 | 0.244949 |
| Day 2 | 8 | 10 | 6 | 9 | 10 | 8.6 | 86 | 1.67332 | 0.748331 |
| Day 3 | 10 | 10 | 10 | 10 | 10 | 10 | 100 | 0 | 0 |
| Day 4 | 10 | 10 | 10 | 10 | 10 | 10 | 100 | 0 | 0 |
| Day 1 | 0 | 2 | 0 | 0 | 0 | 0.4 | 4 | 0.894427 | 0.4 |
| Day 2 | 7 | 7 | 1 | 0 | 6 | 4.2 | 42 | 3.420526 | 1.529706 |
| Day 3 | 10 | 10 | 7 | 10 | 10 | 9.4 | 94 | 1.341641 | 0.6 |
| Day 4 | 10 | 10 | 10 | 10 | 10 | 10 | 100 | 0 | 0 |

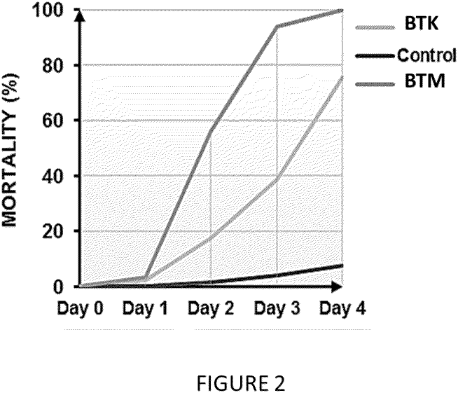

The effect of BTK standard and BTM treatment on average mortality (%) during 4 days post inoculation of Spodoptera frugiperda larvae, is presented in FIG. 2. As shown, the LT₅₀ for BTK standard was found to be 76.6 hr, whereas the LT₅₀ for BTM was found to be 47.4 hr, indicating superior efficacy for BTM product against Spodoptera frugiperda, in addition to its potency against Spodoptera eridania.

### Example 6- Toxic Activity of BTM Against Spodoptera, Heliothis, Plutella

Table 8 hereinbelow, summarizes the toxic activity of BTM against the examined pests, compared to the commercial standards; BTK and BTA.

TABLE 8

| Examined Pest | Relevant plant | Commercial standard (St.) | Toxic activity of BTM [ITU mg⁻¹] | % Toxic activity of BTM compared to the commercial St. |
|---|---|---|---|---|
| Spodoptera eridania | Cotton, soybeans, beets, cabbage, carrots, tomatoes, potatoes, peanuts, avocados, citrus fruits | BTK | 157,397 | 1560% |
| Spodoptera frugiperda | Corn, cotton, rice | BTK | 29,042 | 440% |
| Heliothis virescens | Cotton, tobacco, peas | BTK | 17,134 | 107% |
| Plutella xylostella | Cauliflower | BTA | 508 | 111% |

### Example 7- Toxic Activity of BTM Against Tuta Absoluta

In a first trial, tomato plants exhibiting severe damage by Tuta absoluta moth were divided to the following treatment groups:
1. Control - water spray
2. BTK 0.25% v/v
3. BTK 0.5% v/v
4. BTM 0.25% v/v
5. BTM 0.5% v/v

Each treatment group included 3 plants in 4-5 rectangular pots. Total volume treatment was 5 liter per treatment.

In a second trial, tomato plants with no infestation signs of Tuta absoluta were divided to the following treatments groups:
1. Control - water spray
2. BTK 0.25% v/v
3. BTM 0.25% v/v

Each treatment group included 3 plants in 4-5 rectangular pots. Total volume treatment was 3 liter per treatment. Treatments were applied twice at 1 week interval.

In a third independent trial, tomato plants with infestation signs of Tuta absoluta were divided to the following treatments:
1. Control
2. BTK 0.25% v/v
3. BTM 0.25% v/v

Each treatment group included 3 rows, 15 pots per row and 2-3 plants per pot. Total plants per treatment was about 90-125 plants. Treatments were applied twice at 1 week interval. Infestation rates were examined once a week.

An assessment method was used to evaluate the presence and severity of gallery marks on leaves. It assigns a grade to indicate the level of infection. The grades range from 0 to 10 and represent the following:
0: No presence of gallery marks at all.
1: Initial presence of gallery signs.
2: Presence of small galleries.
3: Presence of large galleries.
4: All the leaves are infected with small galleries.
5: All the leaves are infected with large galleries.
6: Most of the leaves are infected, but less than 50% of each leaf is infected.
7: All the leaves are infected, but less than 50% of each leaf is infected.
8: Most of the leaves are infected, and at least 50% of each leaf is infected.
9: All the leaves are infected, and more than 50% of each leaf surface is infected.
10: All the leaves are infected, and their entire area is covered with gallery marks.
Statistical analysis was performed to all 3 trials combined together by JMP16.

TABLE 9

Mean score and Std error of damage by Tuta absoluta.

| Time (days) | treatment | N | Mean (damaged young leaf) | Mean (damaged mature leaf) | Std Err (damaged young leaf) | Std Err (damaged mature leaf) |
|---|---|---|---|---|---|---|
| 0 | BTK | 48 | 1.31A | - | 0.21 | - |
| | Control | 18 | 1.94A | - | 0.35 | - |
| | BTM | 29 | 1.14A | - | 0.15 | - |
| 7 | BTK | 151 | 2.69B | 5.33A | 0.13 | 0.14 |
| | Control | 79 | 3.99A | 5.42A | 0.24 | 0.24 |
| | BTM | 95 | 2.48B | 3.48B | 0.13 | 0.15 |
| 14 | BTK | 139 | 3.07A | 4.82B | 0.16 | 0.15 |
| | Control | 47 | 3.67A | 5.85A | 0.24 | 0.27 |
| | BTM | 100 | 2.24B | 3.42C | 0.11 | 0.15 |
| 28 | BTK | 166 | 5.46B | 5.55B | 0.09 | 0.10 |
| | Control | 115 | 6.67A | 7.22A | 0.15 | 0.14 |
| | BTM | 105 | 4.41C | 4.51C | 0.11 | 0.11 |

The mean and Std error of Tuta absoluta damage to young and mature tomato plant leaves following treatment over 4 weeks trials in 3 independent greenhouse experiments are summarized in table 9. Each time point followed by different letter is significantly different from the other treatment within young or mature leaf (as examined by Tukey HSD).

One-way analysis of damage index by time revealed the following results:
Young leaf:
   Time 0: F=3.5, p>0.5; Time 7: F=21.02 DF=2,316, P<0.0001; Time 14: F=14, DF=2,2, P<0.0001; Time 28: F=81.2, DF=2,382, P<0.0001
Mature leaf:
   Time 7: F=3.39 DF=2,278, P<0.0001; Time 14: F=36.86, DF=2,280, P<0.0001; Time 28: F=117.17, DF=2,382, P<0.0001.

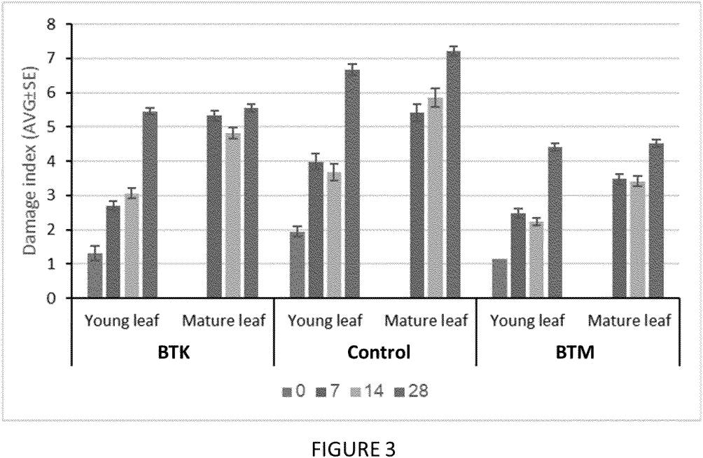

FIG. 3 illustrates the summary results of the damage index in young and mature tomato plant leaves exposed to Tuta absoluta. A significant amelioration in damage index was observed in BTM treatment compared to BTK treatment and compared to control, in all the examined time points: 7, 14, and 28 days.

A number of implementations have been described. Nevertheless, it will be understood that various modifications may be made without departing from the spirit and scope of the disclosure. For example, various forms of the materials shown above may be used, with steps re-ordered, added, or removed. Accordingly, other implementations are within the scope of the following claims.

The examples presented herein are intended primarily for purposes of illustration of the invention for those skilled in the art, and to illustrate potential and specific implementations of the present disclosure. No particular aspect or aspects of the examples are necessarily intended to limit the scope of the present invention.

The figures and descriptions of the present invention have been simplified to illustrate elements that are relevant for a clear understanding while eliminating, for purpose of clarity, other elements. Those of ordinary skill in the art may recognize, however, that these sorts of focused discussions would not facilitate a better understanding of the present disclosure, and therefore, a more detailed description of such elements is not provided herein.

Unless otherwise indicated, all numbers expressing lengths, widths, depths, or other dimensions and so forth used in the specification and claims are to be understood in all instances as indicating both the exact values as shown and as being modified by the term “about.” As used herein, the term “about” refers to a variation from the nominal value. Accordingly, unless indicated to the contrary, the numerical parameters set forth in the specification and attached claims are approximations that may vary depending upon the desired properties sought to be obtained.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some ofthe steps described above may be order independent, and thus can be performed in an order different from that described.

1. A pesticide composition comprising at least two proteins selected from the group consisting of: CrylIa10, Cry2Ab1, Cry9Ea1, and the amino-acid sequence set forth in SEQ ID NO: 6 (M100 Vip3Aa79).

2. The pesticide composition of claim 1, comprising CrylIa10, Cry2Ab1, Cry9Ea1, and the amino-acid sequence set forth in SEQ ID NO: 6.

3. The pesticide composition of claim 1, further comprising at least one protein selected from the group consisting of: Chitinase, and Endochitinase.

4. The pesticide composition of claim 2, further comprising Chitinase, and Endochitinase.

5. A nucleic-acid construct comprising a nucleic-acid sequence encoding at least two proteins selected from: CrylIa10, Cry2Ab1, Cry9Ea1, and the amino-acid sequence set forth in SEQ ID NO: 6.

6. The nucleic acid construct of claim 5, comprising a nucleic-acid sequence encoding CrylIa10, Cry2Ab1, Cry9Ea1, and a nucleic-acid sequence encoding the amino-acid sequence set forth in SEQ ID NO: 6.

7. The nucleic acid construct of claim 6, wherein said nucleic-acid sequence encoding the amino-acid sequence set forth in SEQ ID NO: 6, comprises the nucleic-acid sequence set forth in SEQ ID NO: 12.

8. The nucleic acid construct of claim 7, further comprising a nucleic-acid sequence encoding Chitinase, Endochitinase, and both.

9. The pesticide composition of claim 1, further comprising a bacterial cell or portion thereof.

10. The pesticide composition of claim 2, further comprising a bacterial cell or portion thereof.

11. The pesticide composition of claim 10, wherein said portion thereof is selected from the group consisting of: an endospore, a crystal protein, an endospore-crystal protein complex, and any combination thereof.

12. The pesticide composition of claim 11, wherein said bacterial cell belongs to Bacillus Thuringiensis specie.

13. The pesticide composition of claim 12, wherein said bacterial cell is M100 strain of said Bacillus Thuringiensis specie.

14. A method of controlling a pest, the method comprising steps of contacting the pest with an effective amount of the pesticide composition comprising at least two proteins selected from the group consisting of: CrylIa10, Cry2Ab 1, Cry9Ea1, and the amino-acid sequence set forth in SEQ ID NO: 6 (M100 Vip3Aa79).

15. The method of claim 14, wherein said composition comprises CrylIa10, Cry2Ab1, Cry9Ea1, the amino-acid sequence set forth in SEQ ID NO: 6 (M100 Vip3Aa79), Chitinase, and Endochitinase.

16. The method of claim 15, wherein said composition further comprises a bacterial cell or portion thereof.

17. The method of claim 15, wherein said pest is a lepidopteran pest.

18. The method of claim 17, wherein said pest is selected from the group consisting of: False codling moth, Carob moth, Darkling beetle, Spodoptera littoralis, Pine processionary moth, Pistachio processionary moth, Spodoptera Frugiperda, Spodoptera eridania, Heliothis virescens, Plutella xylostella, and Tuta absoluta.

19. The method of claim 14, wherein said effective amount is in the range of 500-160,000 ITU/mg.

20. The method of claim 14, wherein said contacting is for less than 4 days.


SEQUENCE LISTING (12 sequences)

SEQ ID NO: 1 - protein, 1227 aa, Bacillus thuringiensis
LTSNRKNENEIINALSIPAVSNHSAQMDLSLDARIEDSLCIAEGNNINPLVSASTVQTGINIAGRILGVLGVPFAGQLASFYSFLVGELWPRGRDQWEIFLEHVEQLINQQITENARNTALARLQGLGDSFRAYQQSLEDWLENRDDARTRSVLYIQYIALELDFLNAMPLFAIRNQEVPLLMVYAQAANLHLLLLRDASLFGSEFGLTSQEIQRYYERQVERTRDYSDYCVEWYNTGLNSLRGTNAASWVRYNQFRRDLTLGVLDLVALFPSYDTRTYPINTSAQLTREVYTDAIGTVHPHQAFASTTWYNNNAPSLSAIEAAVIRSPHLLDFPEQLTIYSTLSRWSNTQYMNIWVGHRLESRTIGGSLNTSTQGSTNTSINPVRLQFTARDVYRTESLAGLNIFLTQPVNGVPWVRFNWRNPLNSLRGSLLYTIGYTGVGTQLQDSETELPPETTERPNYESYSHRLSHIGLISSSHVRALVYSWTHRSADRTNTIGPNRITQIPLVKALNLHSGATVVRGPGFTGGDILRRTNTGTFGDIRLNINVPLSQRYRVRIRYASTTDLQFFTRINGTTVNIANFSRTMNRGDNLESRSFRTAGFSTPFNFSNAQSTFTLGAQSFSNQEVYIDRVEFVPAEVTFEAEYDLERAQEAVNALFTNTNPRRLKTDVTDYHIDQVSNLVAYLSDEFCLDEKRELLEKVKYAKRLSDERNLLQDPNFTSINKQPDFISTNEQSNFTSIHEQSEHGWWGSENITIQEGNDVFKENYVTLPGTFNECYPTYLYQKIGESELKAYTRYQLRGYIEDSQDLEIYLIRYNAKHETLDVPGTESVWPLSVESPIGRCGEPNRCAPHFEWNPDLDCSCRDGEKCAHHSHHFSLDIDIGCTDLHENLGVWVVFKIKTQEGHARLGNLEFIEEKPLLGEALSRVKREEKKWRDKREKLQLETKRVYTEAKEAVDALFVDSQYDRLQADTNIGMIHAADKLVHRIREAYLSELPVIPGVNAEIFEELEGHIITAISLYDARNVVKNGDFNNGLTCWNVKGHVDVQQSHHRSDLVIPEWEAEVSQAVRVCPGRGYILRVTAYKEGYGEGCVTIHEIENNTDELKFKNCEEEEVYPTDTGTCNDYTAHQGTAACNSRNAGYEDAYEVDTTASVNYKPTYEEETYTDVRRDNHCEYDRGYVNYPPVPAGYVTKELEYFPETDTVWIEIGETEGKFIVDSVELLLMEE

SEQ ID NO: 2 - protein, 719 aa, Bacillus thuringiensis
MKSKNQNMYQSLSSNTTVDKNFTNSLENNTNMELQNINYEDCLRMSEYEGIEPFVSVSTIQTGIGIAGKILGTLGVPFAGQVASLYSFILGELWPKGKSQWEIFMEHVEEIINQKISTYARSKALTDLKGLGDALAVYHESLESWVGNRNNTRARSVVKSQYIALELMFVQKLPSFAVSGEEVTLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTIHPHPSFTSTTWYNNNAPSFSAIEAAVVRNPHLLDFLEQVTIYSLLSRWSNTQYMNMWGGHKLEFRTIGGTLNTSTQGSTNTAINPVTLPFTSRDVYRTESLAGLNLFLTQPVNGVPRVDFHWKFVTHPIASDNFYYPGYAGIGTQLQDSENELPSEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFSDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQIHIERNM

SEQ ID NO: 3 - protein, 590 aa, Bacillus thuringiensis
MDGVEKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNNDGVNDNGARFSDINIGNVVASSNSDVPLDINVTLNSGTQFDLMNIMLVPTNISPLY

SEQ ID NO: 4 - protein, 791 aa, Bacillus thuringiensis
MNMNNNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEVSNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 5 - protein, 769 aa, Bacillus thuringiensis
MKSMKKKLASVVTCTLLAPMFFNGNVNTAYADNKINQDSSKQEYQQKEMDRKGLLGYYFKDKDFSNLTMFSPTRYNTLIYDQQTANKLLDKKQQEYQSIRWIGLIQSNKTGDFTFELSDDECAIIEMDGKVISNKGKEKQVVHLEKGKLIPIKIEYQLDEPLNIDDEKFKGFKLLKVDNQKQLHQVQQDELRNPEFNKKESQEFLAKASKINLFTKKIKRDIDEGTDTDGDSIPDMWEENGYTIQNRIAVKWNDSLASKGYTKFVSNPLDSHTVGDPYTDYEKASRDLDLSNAKETFNPLVAAFPSVNVSMEKVILSPNKNLSNSVESHSSTNWSYTNTEGASVEAGIGPKGFSFGVSANYQHSETVAQEWGASIGDTTQLNTASAGYLNANVRYNNVGTGAIYDVKPTTSFVLEKNTIATITAKSNSTALSISPGESYPKKGQNGIAITSMDDFNSHPITLNKKQLDQVLTNNPIMLETDQTDGIYKIKDTHGNIVTGGTWNGVTQQIKAKTASIIVDDGKQVAEKRVAAKDYAYPEDKTPSLTLKDALKLSFPEEIKETDGLLYYNNNPIYESSVMTYLDENTAKEVKKQINDKTGEFKDVQHLYAVKLTPKMNFTIKVPVAYDTAKQAVNLGGDNPWGAKGLLGTWVNAMVVDNSGDKAYKRVEPGYLLSPTLEFSEGSLDNLKKNYSFYVSMYVKSDKPFTLRINAGPYSTKRTIEASNDFKRVDIPAFYIEGFPIDTIRLEGSDYPSAIWWKDVSITEVSAVKK

SEQ ID NO: 6 - protein, 791 aa, Bacillus thuringiensis
MNMNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 7 - DNA, 3684 bp, Bacillus thuringiensis
  source 1..3684: mol_type = genomic DNA
ttgacttcaaataggaaaaatgagaatgaaattataaatgccttatcgattccagctgtatcgaatcattccgcacaaatggatctatcgctagatgctcgtattgaggattctttgtgtatagccgaggggaataatatcaatccacttgttagcgcctcaacagtccaaacgggtattaacatagctggtagaatactaggtgtattaggcgtaccgtttgctggacaactagctagtttttatagttttcttgtcggtgaattatggccccgcggcagagatcagtgggaaattttcttagaacatgtcgaacaacttataaatcaacaaataacagaaaatgctaggaatacggcacttgctcgattacaaggtttaggagattcctttagagcctatcaacagtcacttgaagattggctagaaaaccgtgatgatgcaagaacgagaagtgttctttatatccaatatatagctttagaacttgattttcttaatgcaatgccgcttttcgcaattagaaaccaagaagttccattattgatggtatatgctcaagctgcaaatttacacctattattattgagagatgcctctctttttggtagtgaatttgggcttacatcgcaggaaattcaacgctattatgagcgccaagtggaacgaacgagagattattccgactattgcgtagaatggtataatacaggtctaaatagcttgagagggacaaatgccgcaagttgggtacggtataatcaattccgtagagatctaacgttaggagtattagatctagtggcgctattcccaagctatgacactcgcacttatccaataaatacgagtgctcagttaacaagggaagtttatacagacgcaattgggactgtacatccgcatcaagcttttgcaagtacgacttggtataataataatgcaccttcgctctctgccatagaggctgcggttatccgaagcccgcatctacttgattttccagaacaacttacaatttacagcacattaagtcgatggagtaacactcagtatatgaatatatgggtaggtcatagacttgaatctcgaacaataggagggtcattaaatacctcgacacaaggatctaccaatacttctattaatcctgtaagattacagtttacggctcgagacgtttataggactgaatcattggcagggctaaatatatttttaactcaacctgttaatggggtaccttgggttagatttaattggagaaatcccctgaattctcttagaggtagccttctctatactatagggtatactggagttgggacgcaattacaagattcagaaactgaattaccaccagaaacaacagaacgaccaaattatgaatcttacagtcatagattatctcatataggactcatttcatcatctcatgtgagagcattggtatattcttggacgcaccgtagtgcagatcgtacaaatacaattggaccaaatagaattacacaaataccattggtaaaagcacttaaccttcattcaggtgctactgttgttagagggccaggatttacaggtggggatatccttcgtagaacgaatactggtacatttggagatatacgtttaaatattaatgtgccattatcccaaagatatcgcgtaaggattcgttatgcttctactacagatttacaatttttcacgagaattaatggaaccactgttaatattgctaatttctcaagaactatgaataggggggataatttagaatctagaagttttagaactgcaggatttagtactccttttaatttttcaaatgcccaaagcacattcacattgggtgctcagagtttttcaaatcaggaagtttatatagatagagtcgaatttgttccggcagaggtaaccttcgaagcagaatatgatttagaaagagcgcaagaggcggtgaatgctctgtttactaatacgaatccaagaagattgaaaacagatgtgacagattatcatattgatcaagtatccaatctggtagcgtatttatcggatgaattctgcttagatgaaaagagagaattacttgagaaagtgaaatatgcgaaacgactcagtgatgaaagaaacttactccaagatccaaacttcacatccatcaataagcaaccagacttcatatctactaatgagcaatcgaatttcacatctatccatgaacaatctgaacatggatggtggggaagtgagaacatcacaatccaggaaggaaatgacgtatttaaagagaattacgtcacactaccggggacttttaatgagtgttatccgacgtatttatatcaaaaaataggagagtcggaattaaaagcttatactcgctaccaattaagaggttatattgaagatagtcaagatttagagatatatttgattcgttataatgcgaaacatgaaacattggatgttccaggtaccgagtccgtatggccgctttcagttgaaagcccaatcggaaggtgcggagaaccgaatcgatgcgcaccacattttgaatggaatcctgatctagattgttcctgcagagatggagaaaaatgtgcgcatcattcccatcatttctctttggatattgatattggatgcacagacttgcatgagaatctaggcgtgtgggtggtattcaagattaagacgcaggaaggtcatgcaagactagggaatctggaatttattgaagagaaaccattattaggagaagcactgtctcgtgtgaagagggaagagaaaaaatggagagacaaacgtgaaaaactacaattggaaacaaaacgagtatatacagaggcaaaagaagctgtggatgctttattcgtagattctcaatatgatagattacaagcggatacaaacattggcatgattcatgcggcagataaacttgttcatcgaattcgagaggcgtatctttcagaattacctgttatcccaggtgtaaatgcggaaatttttgaagaattagaaggtcacattatcactgcaatctccttatacgatgcgagaaatgtcgttaaaaatggtgattttaataatggattaacatgttggaatgtaaaagggcatgtagatgtacaacagagccatcatcgttctgaccttgttatcccagaatgggaagcagaagtgtcacaagcagttcgcgtctgtccggggcgtggctatatcctccgtgtcacagcgtacaaagagggatatggagagggctgcgtaacgatccatgaaatcgagaacaatacagacgaactaaaatttaaaaactgtgaagaagaggaagtgtatccaacggatacaggaacgtgtaatgattatactgcacaccaaggtacagcagcatgtaattcccgtaatgctggatatgaggatgcatatgaagttgatactacagcatctgttaattacaaaccgacttatgaagaagaaacgtatacagatgtacgaagagataatcattgtgaatatgacagagggtatgtgaattatccaccagtaccagctggttatgtgacaaaagaattagaatacttcccagaaacagatacagtatggattgagattggagaaacggaaggaaagtttattgtagatagcgtggaattactcctcatggaagaatag

SEQ ID NO: 8 - DNA, 2160 bp, Bacillus thuringiensis
  source 1..2160: mol_type = genomic DNA
atgaaatctaagaatcaaaatatgtatcaaagcttgtctagcaatacgacagttgataaaaactttacaaattcactagaaaacaacacaaatatggaattacaaaatattaattatgaagattgtttgagaatgtctgagtatgaaggtatagagccgtttgttagtgtatcaacaattcaaacaggtattggtattgcgggtaaaatacttggtaccctaggcgttccttttgcaggacaagtagctagtctttatagttttatcttaggtgagctatggcctaaggggaaaagccaatgggaaatctttatggaacatgtagaagagattattaatcaaaaaatatcaacttatgcaagaagtaaagcacttacagacttgaaaggattaggagatgccttagctgtctaccatgaatcgctggaaagttgggttggaaatcgtaataacacaagggctaggagtgttgtcaagagccaatatatcgcattagaattgatgttcgttcagaaactaccttcttttgcagtgtctggagaggaggtaacattattaccgatatatgcccaagctgcaaatttacatttgttgctattacgagatgcgtctatttttggaaaagagtggggattatcatcttcagaaatttcaacattttataaccgtcaagtcgaacgagcaggagattattccgaccattgtgtgaaatggtatagcacaggtctaaataacttgaggggtacaaatgccgaaagttgggtacgatataatcaattccgtagagacatgactttaatggtactagatttagtggcactatttccaagctatgatacacaaatgtatccaattaaaactacagcccaacttacaagagaagtatatacagacgcaattgggacaatacatccgcatccaagttttacaagtacgacttggtataataataatgcaccttcgttctctgccatagaggctgctgttgttcgaaacccgcatctactcgattttctagaacaagttacaatttacagcttattaagtcgatggagtaacactcagtatatgaatatgtggggaggacataaactagaattccgaacaataggaggaacgttaaatacctcaacacaaggatctactaatactgctattaatcctgtaacattaccgttcacttctcgagacgtctataggactgaatcattggcagggctgaatctatttttaactcaacctgttaatggagtacctagggttgattttcattggaaattcgtcacacatccgatcgcatctgataatttctattatccagggtatgctggaattgggacgcaattacaggattcagaaaatgaattaccatctgaagcaacaggacagccaaattatgaatcttatagtcatagattatctcatataggactcatttcagcatcacatgtgaaagcattggtatattcttggacacatcgtagtgcagatcgtacaaatacaattgagccaaatagcattacacaaataccattagtaaaagcgttcaatctgtcttcaggtgccgctgtagtgagaggaccaggttttacaggtggggatatccttcgaagaacgaatactggtacatttggggatatacgagtaaatattaatccaccatttgcacaaagatatcgcgtgaggattcgctatgcttctactacagatttacaattccatacgtcaattaacggtaaagctattaatcaaggtaatttttcagcaactatgaatagaggagaggacttagactataaaacctttagaactgtaggctttaccactccatttagcttttcagatgtacaaagtacattcacaataggtgcttggaacttctcttcaggtaacgaagtttatatagatagaattgaatttgttccggtagaagtaacatatgaggcagaatatgattttgaaaaagcgcaagagaaggttactgcactgtttacatctacgaatccaagaggattaaaaacagatgtaaaggattatcatattgaccaggtatcaaatttagtagagtctctatcagatgaattctatcttgatgaaaagagagaattattcgagatagttaaatacgcgaagcaaatccatattgagcgtaacatgtag

SEQ ID NO: 9 - DNA, 1773 bp, Bacillus thuringiensis
  source 1..1773: mol_type = genomic DNA
atggacggagtggaaaaaaataatcatagtttatacctagatcctattgttggaactgtggctagttttctgttaaagaaagtggggagtcttgttggaaaaaggatactaagtgagttacggaatttaatatttcctagtggtagtacaaatctaatgcaagatattttaagagagacagaaaaattcctgaatcaaagacttaatacagacactcttgcccgtgtaaatgcggaattgacagggctgcaagcaaatgtagaagaatttaatcgacaagtagataattttttgaaccctaaccgaaacgctgttcctttatcaataacttcttcagttaatacaatgcaacaattatttctaaatagattaccccagttccagatgcaaggataccaactgttattattacctttatttgcacaggcagccaatttacatctttcttttattagagatgttattctaaatgcagatgaatggggaatttcagcagcaacattacgtacgtatcgagattacttgaaaaattatacaagagattactctaactattgtataaatacgtatcaaagtgcgtttaaaggtttaaacactcgtttacacgatatgttagaatttagaacatatatgtttttaaatgtatttgaatatgtatctatctggtcgttgtttaaatatcaaagtcttctagtatcttccggtgctaatttatatgcaagtggtagtggaccacagcagacccaatcatttacttcacaagactggccatttttatattctcttttccaagttaattcaaattatgtgttaaatggatttagtggtgctaggctttctaataccttccctaatatagttggtttacctggttctactacaactcacgcattgcttgctgcaagggttaattacagtggaggaatttcgtctggtgatataggtgcatctccgtttaatcaaaattttaattgtagcacatttctccccccattgttaacgccatttgttaggagttggctagattcaggttcagatcgggagggcgttgccaccgttacaaattggcaaacagaatcctttgagacaactttagggttaaggagtggtgcttttacagctcgcggtaattcaaactatttcccagattattttattcgtaatatttctggagttcctttagttgttagaaatgaagatttaagaagaccattacactataatgaaataagaaatatagcaagtccttcaggaacacctggtggagcacgagcttatatggtatctgtgcataacagaaaaaataatatccatgctgttcatgaaaatggttctatgattcatttagcgccaaatgactatacaggatttactatttcgccgatacatgcaactcaagtgaataatcaaacacgaacatttatttctgaaaaatttggaaatcaaggtgattctttaaggtttgaacaaaacaacacgacagctcgttatacgcttagagggaatggaaatagttacaatctttatttaagagtttcttcaataggaaattccactattcgagttactataaacggtagggtatatactgctacaaataacgatggagttaatgataatggagctcgtttttcagatattaatatcggtaatgtagtagcaagtagtaattctgatgtaccattagatataaatgtaacattaaactccggtactcaatttgatcttatgaatattatgcttgtaccaactaatatttcaccactttattaa

SEQ ID NO: 10 - DNA, 2376 bp, Bacillus thuringiensis
  source 1..2376: mol_type = genomic DNA
atgaacatgaacaataataatactaaattaagcacaagagccttaccaagttttattgattattttaatggcatttatggatttgccactggtatcaaagacattatgaacatgatttttaaaacggatacaggtggtgatctaaccctagatgaaattttaaagaatcagcagttactcaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaactcgatgcgataaatacgatgcttcgggtatatctacctaaaattacctctatgttgagtgatgtaatgaaacaaaattatgcgctaagtctgcaaatagaatacttaagtaaacaattgcaagagatttctgataagttagatattattaatgtaaatgtacttattaactctacacttactgaaattacacctgcgtatcaaaggattaaatatgtgaacgaaaaatttgaggaattaacttttgctacagaaactagttcaaaagtaaaaaaggatggctctcctgcagatattcttgatgagttaactgagttaactgaactagcgaagagtgtaacaaaaaatgatgtggatggttttgaattttaccttaatacattccacgatgtaatggtaggaaataatttattcgggcgttcagctttaaaaactgcatcggaattaattactaaagaaaatgtgaaaacaagtggcagtgaggtcggaaatgtttataacttcttaattgtattaacagctctgcaagcaaaagcttttcttactttaacaacatgccgaaaattattaggcttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattggttgggtttgaagttagtaatgattcaattacagtattaaaagtatatgaggctaagctaaaacaaaattatcaagttgataaggattccttatcggaagttatttatggcgatatggataaattattgtgcccagatcaatctgaacaaatctattatacaaataacatagtatttccaaatgaatatgtaattactaaaattgatttcactaaaaaaatgaaaactttaagatatgaggtaacagcgaatttttatgattcttctacaggagaaattgacttaaataagaaaaaagtagaatcaagtgaagcggagtatagaacgttaagtgctaatgatgatggagtgtatatgccgttaggtgtcatcagtgaaacatttttgactccgattaatgggtttggcctccaagctgatgaaaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattactaaacgttttactacaggaactgatttaaagggagtgtatttaattttaaaaagtcaaaatggagatgaagcttggggagataactttattattttggaaattagtccttctgaaaagttattaagtccagaattaattaatacaaataattggacgagtacgggatcaactaatattagcggtaatacactcactctttatcagggagggcgaggaattctaaaacaaaaccttcaattagatagtttttcaacttatagagtgtatttttctgtgtccggagatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaagtaa

SEQ ID NO: 11 - DNA, 2310 bp, Bacillus thuringiensis
  source 1..2310: mol_type = genomic DNA
atgaaaagtatgaagaaaaagctggcaagtgttgtaacctgtaccttattagcccctatgttttttaatgggaatgtaaatactgcttacgcggacaacaaaatcaatcaggattcttcaaagcaggaataccaacagaaagagatggatcgaaaaggattgcttgggtattattttaaagataaagattttagtaaccttactatgttttcaccaacacgttataatacccttatttatgaccaacaaacagcgaataaattattagataaaaaacaacaagaatatcagtctattcgctggattggtttgattcagagtaacaaaacaggtgacttcacatttgaattatcagatgatgaatgtgcgattatagaaatggatgggaaagtgatttctaataaaggaaaagaaaagcaagttgtccatttagagaaaggaaaattaattccaattaaaattgagtatcaattagacgagccattaaatatcgatgatgaaaaattcaaagggtttaaattattgaaagtagataatcaaaaacaactacatcaagtacaacaagatgaattaagaaaccctgaatttaacaaaaaagagtcacaggaatttttagcgaaagcatcgaaaataaatctttttacgaaaaaaattaagagggacattgatgagggaacggatacagacggtgactctattccggatatgtgggaagaaaatgggtatacgattcaaaatagaatcgctgttaaatggaatgactcgctagcaagtaaagggtatacaaaatttgtttctaatccccttgacagtcacacagttggtgacccttatactgactatgaaaaagcatcgagagatttagatttgtcaaatgcaaaagaaacatttaacccattggtagctgcttttccaagcgtgaatgttagcatggaaaaggtgatattatctccaaataaaaacttatctaatagtgtagaatcgcactcatctacgaattggtcttataccaatactgaaggagcatctgttgaagctgggattgggcctaaaggtttttcttttggggtaagtgcaaactatcaacattctgaaacagttgcacaggaatggggggcatctataggagatactacgcaacttaacactgcgtcagcaggatatttaaatgcaaatgttcgatacaacaatgtgggaacaggagccatttatgatgtaaaacctacaactagctttgtattagaaaagaatacaatcgcaactattacggcaaaatcaaattcaacagctttaagtatatctcccggagaaagttatccgaaaaaaggccaaaatggaattgcgataacatccatggatgattttaactcacatccaattacattaaataaaaaacaattagatcaagtattgactaataaccctattatgttagaaacagaccagacagatgggatttataagataaaagatacacatggtaatattgtcacaggtggcacatggaatggtgtaactcaacaaattaaagcaaaaacagcgtctattattgtggatgacgggaaacaggtagcagaaaaacgtgtggcggcaaaagattatgcttatccagaagataaaacaccgtctttaactttaaaagatgcactgaagctttcattcccagaagagataaaagaaacagatggattattgtactataacaataatccaatttatgaatcgagtgtcatgacctaccttgatgagaatacggcaaaggaagtaaaaaaacaaataaatgataaaacaggggaattcaaggatgtccaacatttatatgctgttaaacttacacccaaaatgaattttactattaaagtaccggttgcgtatgatacagccaaacaggctgttaatcttggaggggataatccatggggggctaaaggattattagggacatgggtaaatgccatggttgtagataattccggagataaagcatataaaagggttgaacctggttatctactttcacctacattggagttttcagaaggttcattagacaatttaaagaaaaattatagtttttatgttagtatgtacgtgaaatcagataaaccatttactctaagaataaatgctggaccctatagtaccaagagaaccatagaagcatcaaatgattttaaacgcgttgatattcctgcgttttatatagaaggatttccgatagacactataagattagaaggttctgactatccatcggcaatatggtggaaagatgtttcaattacagaagtgtcagcagtaaaaaaataa

SEQ ID NO: 12 - DNA, 2376 bp, Bacillus thuringiensis
  source 1..2376: mol_type = genomic DNA
atgaacatgaacaagaataatactaaattaagcacaagagccttaccaagttttattgattattttaatggcatttatggatttgccactggtatcaaagacattatgaacatgatttttaaaacggatacaggtggtgatctaaccctagacgaaattttaaagaatcagcagttactaaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaactcgatgcgataaatacgatgcttcgggtatatctacctaaaattacctctatgttgagtgatgtaatgaaacaaaattatgcgctaagtctgcaaatagaatacttaagtaaacaattgcaagagatttctgataagttggatattattaatgtaaatgtacttattaactctacacttactgaaattacacctgcgtatcaaaggattaaatatgtgaacgaaaaatttgaggaattaacttttgctacagaaactagttcaaaagtaaaaaaggatggctctcctgcagatattcttgatgagttaactgagttaactgaactagcgaaaagtgtaacaaaaaatgatgtggatggttttgaattttaccttaatacattccacgatgtaatggtaggaaataatttattcgggcgttcagctttaaaaactgcatcggaattaattactaaagaaaatgtgaaaacaagtggcagtgaggtcggaaatgtttataacttcttaattgtattaacagctctgcaagcaaaagcttttcttactttaacaacatgccgaaaattattaggcttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattgattgggtttgaaattagtaatgattcaattacagtattaaaagtatatgaggctaagctaaaacaaaattatcaagtcgataaggattccttatcggaagttatttatggtgatatggataaattattgtgcccagatcaatctgaacaaatctattatacaaataacatagtatttccaaatgaatatgtaattactaaaattgatttcactaaaaaaatgaaaactttaagatatgaggtaacagcgaatttttatgattcttctacaggagaaattgacttaaataagaaaaaagtagaatcaagtgaagcggagtatagaacgttaagtgctaatgatgatggggtgtatatgccgttaggtgtcatcagtgaaacatttttgactccgattaatgggtttggcctccaagctgatgaaaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattaataaacgttttactacaggaactgatttaaagggagtgtatttaattttaaaaagtcaaaatggagatgaagcttggggagataactttattattttggaaattagtccttctgaaaagttattaagtccagaattaattaatacaaataattggacgagtacgggatcaactaatattagcggtaatacactcactctttatcagggaggacgagggattctaaaacaaaaccttcaattagatagtttttcaacttatagagtgtatttttctgtgtccggagatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaagtaa